(19) United States
(12) Patent Application Publication
(10) Pub. No.: US 2025/0099673 A1
(43) Pub. Date: Mar. 27, 2025

(54) PUMP ASSEMBLY, DRUG INJECTION APPARATUS HAVING THE SAME AND METHOD FOR DETECTING FLOW PATH BLOCKAGE

(71) Applicants: CAREMEDI CO., LTD., Seoul (KR); SOGANG UNIVERSITY RESEARCH & BUSINESS DEVELOPMENT FOUNDATION, Seoul (KR)

(72) Inventors: Woonsup SHIN, Seoul (KR); Enhua ZHU, Seoul (KR)

(21) Appl. No.: 18/934,796

(22) Filed: Nov. 1, 2024

Related U.S. Application Data
(63) Continuation of application No. PCT/KR2024/012749, filed on Aug. 27, 2024.

(30) Foreign Application Priority Data
Sep. 26, 2023 (KR) 10-2023-0129037

(51) Int. Cl. A61M 5/142; A61M 5/168

(52) U.S. Cl. CPC A61M 5/14212; A61M 5/16854; A61M 2005/16872; A61M 2205/3331; A61M 2205/35

(57) ABSTRACT

A pump assembly according to the present disclosure includes an electroosmotic pump that includes a monitoring chamber that is a subject for pressure measurement, and electrochemically generates a positive pressure and a negative pressure alternately to suck and discharge a transfer subject fluid; and a pressure monitoring unit that is coupled to one side of the monitoring chamber and measures an internal pressure of the monitoring chamber.

### CROSS-REFERENCE TO RELATED APPLICATION

This application is based on and claims priority under 35 U.S.C. § 119 to Korean Patent Application No. 10-2023-0129037, filed on Sep. 26, 2023, in the Korean Intellectual Property Office, the disclosure of which is incorporated by reference herein in its entirety.

### BACKGROUND

The present disclosure relates to a pump assembly, drug injection apparatus having the same and method for detecting flow path blockage.

Depending on the type, purpose and method of treatment, drugs may be administered into the body in a variety of ways, including orally, subcutaneously, and intravenously. A drug injector using a drug pump may automatically inject drugs into the body at desired speed and dose. Therefore, the drug injector using a drug pump may be used in various forms as well as in hospitals or patients' daily living environments.

An insulin pump, commonly called insulin injector, is for diabetic patients who do not secrete insulin or secrete only a small amount of insulin. It is a medical apparatus that plays the same role as the pancreas in controlling blood sugar levels by supplying insulin from the outside into the body at a specific time.

Such an insulin pump is used by insulin-dependent diabetic patients and in a state

of being attached to the patient, the drug may be continuously injected for 24 hours. As described above, since the insulin injector must inject drugs into diabetic patients periodically over a long period of time, technology for miniaturization and automation of the insulin injector is being actively developed for user convenience.

However, as the insulin injector becomes miniaturized and automated, there are cases where it is difficult to actually secure the operation stability of the insulin injector. In most cases, a back pressure caused by various reactions between drugs and biological fluids at an end portion of an insertion tube is a main cause of clogging. In particular, this problem of clogging becomes more serious when an amount of drug injected is small or when drug is injected slowly. Clogging caused by the back pressure increases the internal pressure of the insulin injector, and it is possible to monitor an occurrence of clogging by measuring the internal pressure of the insulin injector.

Therefore, there is a need for an apparatus that may monitor clogging of the insulin injector and continuously inject the drug into the patient while also stably injecting a correct amount of the drug.

### SUMMARY

In order to solve the above-mentioned problems, a technical task of the present is to provide a pump assembly including a pressure monitoring unit that more accurately measures an internal pressure of an electroosmotic pump.

In addition, another technical task of the present disclosure is to provide a drug injection apparatus having a pump assembly capable of measuring an internal pressure.

However, the technical tasks that the present embodiment seeks to accomplish are not limited to the technical tasks described above, and other technical tasks may exist.

According to an aspect of the present disclosure, a pump assembly includes an electroosmotic pump that includes a monitoring chamber that is a subject for pressure measurement, and electrochemically generates a positive pressure and a negative pressure alternately to suck and discharge a transfer subject fluid; and a pressure monitoring unit that is coupled to one side of the monitoring chamber and measures an internal pressure of the monitoring chamber.

According to the present disclosure, an internal pressure may be regulated according to an external pressure, a pressure for sucking and discharging the drug may be regulated according to the internal pressure by controlling the pressure monitoring unit that measures the internal pressure, thereby preventing an operation error due to the difference between the external and internal pressures.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a frontal perspective view of a drug injection apparatus according to an embodiment of the present disclosure;

FIG. 2 is a rear perspective view of a drug injection apparatus according to an embodiment of the present disclosure;

FIG. 3 is an internal plan view of the drug injection apparatus illustrated in FIG. 1;

FIG. 4 is a perspective view of a drug storage unit illustrated in FIG. 3;

FIG. 5 is a perspective view of a pump assembly illustrated in FIG. 3;

FIG. 6 is a plan view illustrating a coupling structure of the pump assembly illustrated in FIG. 5;

FIG. 7 is a block diagram schematically illustrating a configuration of an electroosmotic pump illustrated in FIG. 5;

FIG. 8 is a conceptual diagram schematically illustrating a configuration of a driving unit illustrated in FIG. 7;

FIG. 9 is an example diagram schematically illustrating an operation of the driving unit illustrated in FIG. 8;

FIG. 10 is a plan view illustrating a coupling structure of a pressure monitoring unit illustrated in FIG. 6;

FIG. 11 is a sectional view illustrating a coupling portion of the electroosmotic pump and the pressure monitoring unit;

FIG. 12 is a perspective view of a monitoring chamber frame illustrated in FIG. 10;

FIG. 13 a top perspective view illustrating a pressure sensor frame illustrated in FIG. 10;

FIG. 14 is a rear perspective view of the pressure sensor frame illustrated in FIG. 13;

FIG. 15 is a sectional view taken along cutting line A-A′ illustrated in FIG. 10;

FIG. 16 is a block diagram schematically illustrating a drug injection apparatus according to an embodiment of the present disclosure;

FIG. 17 is a flowchart for explaining a method for detecting clogging of an insertion tube according to an embodiment of the present disclosure;

FIG. 18 is a flowchart for explaining an embodiment of a step of determining whether the flow path is clogged illustrated in FIG. 17; and

FIG. 19 is a flowchart for explaining another embodiment of the step of determining whether the flow path is clogged illustrated in FIG. 17.

### DETAILED DESCRIPTION OF THE EMBODIMENTS

Hereinafter, the present disclosure will be described in detail with reference to the attached drawings. However, the present disclosure may be implemented in various different forms and is not limited to the embodiments described herein. In addition, the attached drawings are only intended to facilitate understanding of the embodiments disclosed in this specification, and the technical idea disclosed in this specification is not limited by the attached drawings. In order to clearly explain the present disclosure in the drawings, parts not related to the description are omitted, and the size, shape, and shape of each component illustrated in the drawings may be modified in various ways. Throughout the specification, identical/similar parts are given identical/similar reference numerals.

The suffixes “assembly”, “unit”, and the like used in the following description for components are given or used interchangeably only for the convenience of writing the specification, and do not have distinct meanings or roles in themselves. In addition, when describing the embodiments disclosed in this specification, if it is determined that a specific description of a related known technology may obscure the gist of the embodiments disclosed in this specification, the detailed description thereof is omitted.

Throughout the specification, when a part is said to be “connected (coupled, contacted, or combined)” with another part, this includes not only a case where it is “directly connected (coupled, contacted, or combined),” but also a case where it is “indirectly connected (coupled, contacted, or combined) with other members therebetween. Additionally, when a part is said to “include (have or provide)” a certain component, this does not mean that it excludes other components, but rather that it may “include (have or provide)” other components, unless otherwise specifically stated.

Terms representing ordinal numbers, such as first, second, etc., used in this specification are used only for the purpose of distinguishing one component from another component and do not limit the order or relationship of the components. For example, the first component of the present disclosure may be named a second component, and similarly, the second component may also be named a first component.

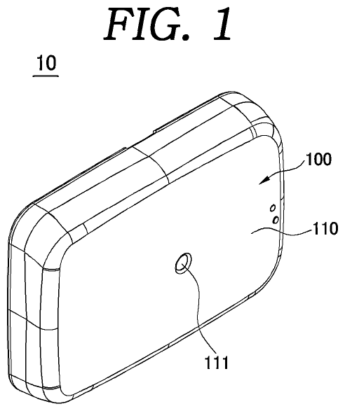

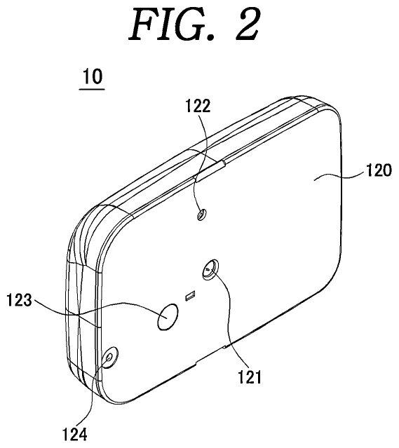

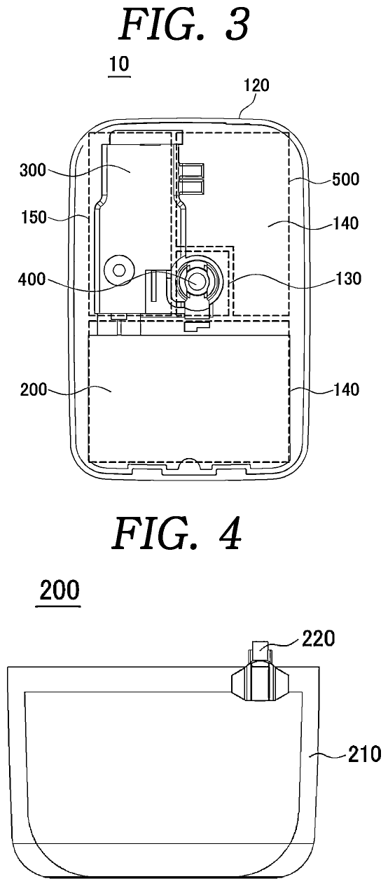

FIG. 1 is a frontal perspective view of a drug injection apparatus according to an embodiment of the present disclosure, FIG. 2 is a rear perspective view of the drug injection apparatus according to an embodiment of the present disclosure, and FIG. 3 is an exploded perspective view of a housing illustrated in FIG. 1.

When describing a patch-type drug injection apparatus 10 according to an embodiment of the present disclosure with reference to FIGS. 1 to 3, the patch-type drug injection apparatus 10 includes a housing 100 and components disposed inside the housing 100.

The housing 100 includes a cover portion 110 and a contact surface 120, and a mounting space is formed between the cover portion 110 and the contact surface 120. The cover portion 110 has rounded corners, and it allows the patch-type drug injection apparatus 10 to remain attached. The contact surface 120 is in a configuration that is attached to an injection target, and an outlet 121 is formed for withdrawing an insertion tube to the outside.

The mounting space is divided into a central region 130 and first to third regions 140, 150, and 160, and the first to third regions 140, 150, and 160 are formed to surround the central region 130. In particular, the mounting space within the housing 100 is divided into the first to third regions 140, 150, and 160, and the central region 130 extending in a direction perpendicular to an insertion direction of the insertion tube. Major components are coupled to each region. In general, a PCB circuit portion is first disposed over the entire or most of a plane of the apparatus, and then the drug storage unit art, pump, etc. are disposed on top thereof, so there is a problem that the thickness of the apparatus is formed thick. However, in order to make the patch-type drug injection device (10) of the present disclosure thin, the region where each component overlaps with each other is minimized.

The components include a drug storage unit 200, a pump assembly 300, an injection assembly 400, and a circuit portion 500, and may be disposed in the central region 130 and the first to third regions 140, 150, and 160 described above. For example, the drug storage unit 200 may be disposed in the first region 140, the pump assembly 300 may be disposed in the second region 150, and the circuit portion 500 may be disposed in the third region 160, the injection assembly 400 may be disposed in the central region 130. Therefore, the drug storage unit 200, the pump assembly 300, and the circuit unit 500 may be disposed to surround the injection assembly 400.

As described above, the injection assembly 400 including the insertion tube is disposed in the central region 130, so that the patch-type drug injection apparatus 10 may maintain a state of being coupled to the user without the insertion tube coming off even when there is a change such as shaking due to the user's movement.

Next, the drug storage unit 200, the pump assembly 300, the injection assembly 400, and the circuit unit 500 will be described in detail.

FIG. 4 is a perspective view of the drug storage unit illustrated in FIG. 3. With reference to FIG. 4, the drug storage unit 200 is described. The drug storage unit 200 stores the drug to be injected, and a thickness thereof changes depending on the amount of drug injected. The drug storage unit 200 may be provided in a flat pouch bag made of polymer. A flange portion 210 in which both surfaces constituting the drug storage unit 200 are joined to each other by a predetermined width may be formed around the drug storage unit 200. The flange portion 210 may limit the expansion of the drug storage unit 200 toward a drug circumference side.

The drug storage unit 200 is configured of release paper, the inside and outside of the drug storage unit 200 are made of materials of which melting temperatures are different from each other, and the melting temperature of the inside material is lower than the melting temperature of the outside material. The drug storage unit 200 is made by forming the flange portion 210 by applying heat to a predetermined temperature at which the inside material melts. When heat is applied to the side thereof, the inside material is bonded together and finished, and the lower side is folded into a W shape and heated. At this time, the inner materials stick to each other and the outer materials do not stick to each other, so they are finished in a wrinkled shape to form the flange portion 210.

In addition, the drug storage unit 200 is coupled with a drug entrance 220 through which the drug enters and exits, so that the drug flows into the drug storage unit 200, or the drug stored in the drug storage unit 200 is discharged to the outside.

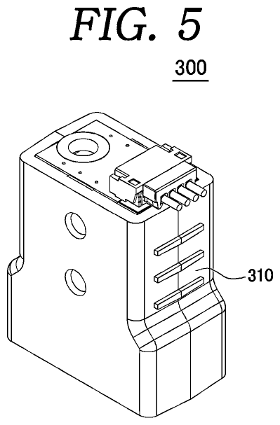

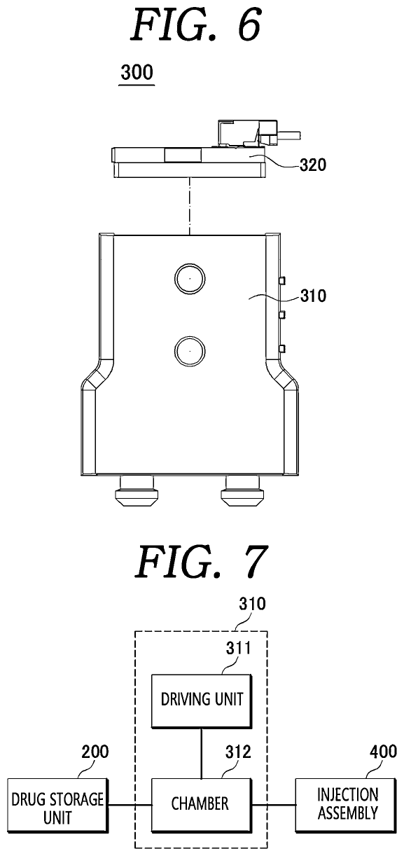

FIG. 5 is a perspective view of the pump assembly illustrated in FIG. 3, and FIG. 6 is a plan view illustrating a coupling structure of the pump assembly illustrated in FIG. 5.

When describing the pump assembly 300 with reference to FIGS. 5 and 6, the pump assembly 300 serves as driving means in the drug injection apparatus 10, and operates as a pump that sucks the drug from the drug storage unit 200, and discharges the drug to the outside.

The pump assembly 300 includes an electroosmotic pump 310 and a pressure monitoring unit 320. The electroosmotic pump 310 includes a monitoring chamber that is a subject for pressure measurement, and electrochemically generates a positive pressure and a negative pressure alternately to suck the drug from the drug storage unit 200 and to discharge the sucked drug into the injection assembly 400.

The pressure monitoring unit 320 measures the internal pressure of the monitoring chamber, and thus, may detect clogging of the flow path of the pump assembly 300 or the drug injection apparatus 10. In other words, the pressure monitoring unit 320 detects a case where the pressure rises abnormally due to flow path clogging.

In addition, the pressure monitoring unit 320 of the present disclosure minimizes a pressure difference between outside and inside of the pump assembly 300 for more accurate pressure measurement, and for this purpose, the pressure monitoring unit 320 also changes the internal pressure according to the external pressure change.

Next, each of the electroosmotic pump 310 and the pressure monitoring unit 320 will be described in detail.

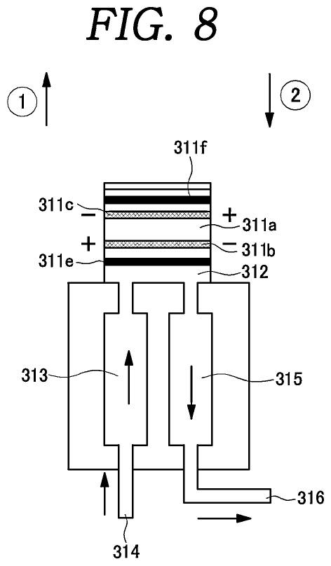

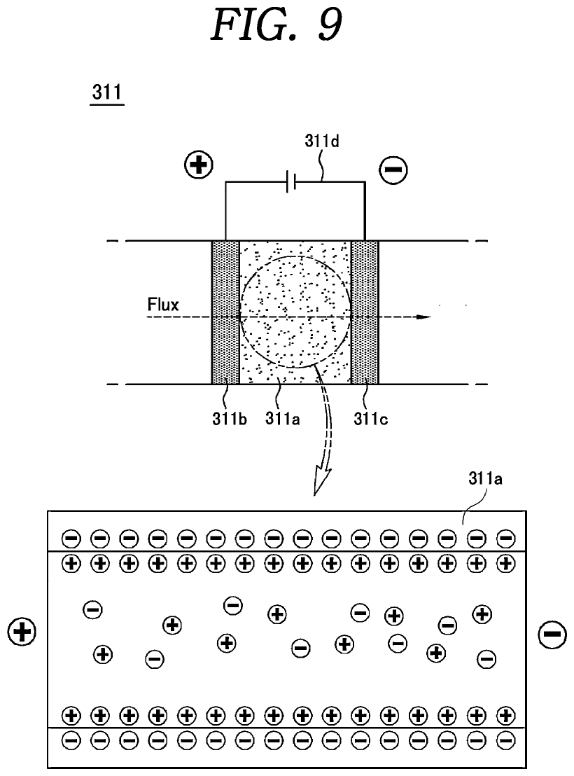

FIG. 7 is a block diagram schematically illustrating a configuration of the electroosmotic pump 310 illustrated in FIG. 6, FIG. 8 is a block diagram schematically illustrating a configuration of a driving unit illustrated in FIG. 7, and FIG. 9 is an example diagram schematically illustrating an operation of the driving unit illustrated in FIG. 8.

The electroosmotic pump 310 will be described in detail with reference to FIGS. 7 and 8. The electroosmotic pump 310 includes a driving unit 311, a transfer chamber 312, suction portions 313 and 314, and discharge portions 315 and 316. The driving unit 311 is electrochemically driven by a control signal to generate the positive pressure and the negative pressure, and sucks and discharges the drug according to the positive pressure and the negative pressure. The transfer chamber 312 receives the drug sucked from the drug storage unit 200 by the driving unit 311. The suction portion 313 delivers the drug to the transfer chamber, the discharge portion 314 outputs the drug from the transfer chamber 312. Here, the drug is a drug that must be injected into a specific patient, and an example of the drug is insulin injected into diabetic patients.

Referring to FIGS. 8 and 9 to describe the driving unit 311 in detail, the driving unit 311 is a pump that uses the movement of a fluid by the electroosmotic phenomenon that occurs when a voltage or a current is applied using electrodes at both ends of a pressure generating portion (membrane) 311a. The driving unit 311 may include a power source 311d that applies the voltage or the current to a first electrode 311b and a second electrode 311c disposed on both sides of the membrane 311a, and a first isolation portion (first diaphragm) 311e and a second isolation portion (first diaphragm) 311f for moving the fluid. In the respective diaphragm 311e and 311f that are provided on one side and the other side of the membrane 311a, the shapes thereof are transformed by the movement of the pumping solution according to the alternate generation of the positive pressure and the negative pressure. Illustratively, the first diaphragm 311e and the second diaphragm 311f deliver absorbs the negative pressure and the positive pressure generated by the driving of the membrane 311a to the transfer target fluid. More specifically, when the negative pressure generates, at least a part of the first diaphragm 311e and the second diaphragm 311f is retracted (when moved in a \textcircled{1} direction), and the transfer target fluid is sucked into the transfer chamber 312. Conversely, when the positive pressure generates, at least a part of the first diaphragm 311e and the second diaphragm 311f is advanced (moved in a \textcircled{2} direction), and the transfer target fluid is discharged from the transfer chamber 312.

As materials of the membrane 311a, silica, glass, and the like are generally used, and when these are immersed in an aqueous solution, a surface thereof is negatively charged. The membrane 311a has numerous paths through which various fluids may pass. When zooming in on one of these, the surface of the fluid path, which has a negative charge (bound anion), may be balanced by mobile cations that have a moveable (+) charge. In this state, when a (+) voltage is applied to the first electrode 311b and a (−) voltage is applied to the second electrode 311c, the negative pressure is generated. This negative pressure causes the fluid inside the driving unit 311 to move in the \textcircled{1} direction. At this time, the drug is sucked through a suction path 314, and flows into the transfer chamber 312 through a suction valve 313. At this time, a discharge valve 315 is closed to prevent the negative pressure from being transmitted to a discharge path 316. Conversely, when the (−) voltage is applied to the first electrode 311b and the (+) voltage is applied to the second electrode 311c, the positive pressure in the opposite direction is generated by a reversible electrochemical reaction. Due to this positive pressure, the fluid inside the driving unit 311 moves in the \textcircled{2} direction. At this time, the drug stored in the transfer chamber 312 is injected into the subject through the discharge valve 315 and the discharge path 316. At this time, the suction valve 313 is clogged to prevent the positive pressure from being transmitted to the suction path 314.

Such a phenomenon is called electroosmotic phenomenon, and the pump that uses this principle is the electroosmotic pump 310. Because the pump assembly 300 of the present disclosure uses the electroosmotic pump 310 as the driving means, it may be manufactured thinner than other driving means such as an electric motor. Furthermore, the overall thickness of the patch-type drug injection apparatus 10 using the pump assembly 300 as the driving means may be manufactured to be thin, thereby capable of being more stably bonded to the skin.

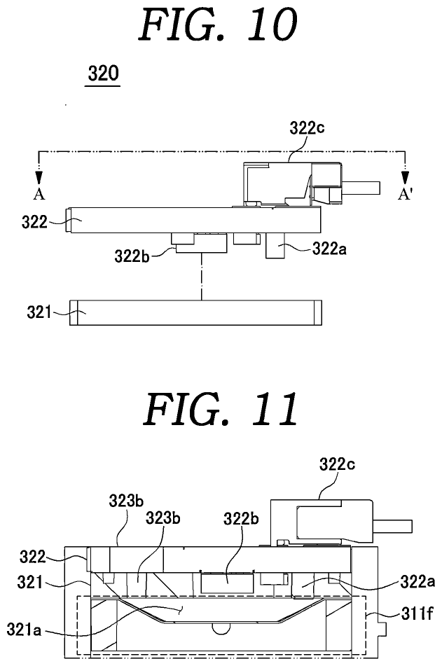

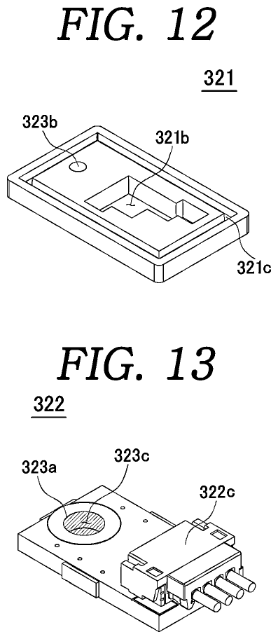

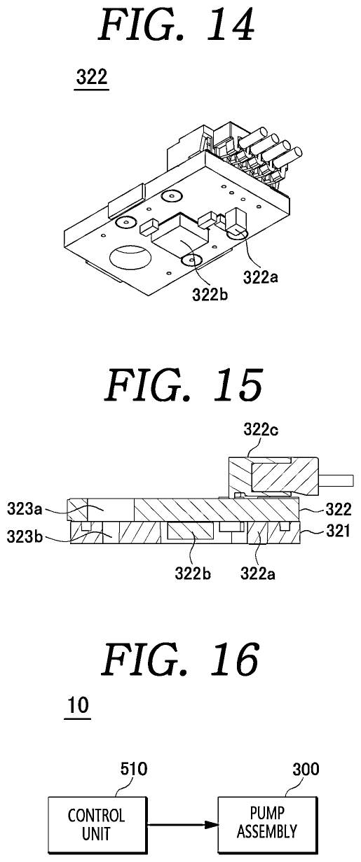

FIG. 10 is a plan view illustrating a coupling structure of the pressure monitoring unit, FIG. 11 is a sectional view illustrating a coupling portion of the electroosmotic pump and the pressure monitoring unit, FIG. 12 is a top perspective view of a monitoring chamber frame illustrated in FIG. 10, FIG. 13 a top perspective view illustrating the pressure sensor frame illustrated in FIG. 10, FIG. 14 is a rear perspective view of the pressure sensor frame illustrated in FIG. 10, and FIG. 15 is a sectional view taken along cutting line A-A′ illustrated in FIG. 10.

The pressure monitoring unit 320 will be described with reference to FIGS. 10 to 15. The pressure monitoring unit 320 is coupled to the electroosmotic pump 310 and measures the internal pressure of the electroosmotic pump 310. The pressure monitoring unit 320 is coupled to the driving unit 311 of the electroosmotic pump 310. Specifically, the pressure monitoring unit 320 is coupled to the second isolation portion 311f (second diaphragm) on the opposite side of the pressure generating portion 311a (membrane) of the driving unit 311. The pressure monitoring unit 320 is coupled to the second isolation portion 311f to finish the monitoring chamber 321a, which is an open space on one side of the second isolation portion 311f, and measure the internal pressure of the monitoring chamber 321a.

In order to perform this role, the pressure monitoring unit 320 includes a monitoring chamber frame 321 and a pressure sensor frame 322.

Referring to FIGS. 11 and 12, the monitoring chamber frame 321 is coupled to the second isolation portion 311f to finish the monitoring chamber 321a, which is a space in which a pressure is measured, and form a communication space 321b to expose the pressure sensor 322a, which will be described later, to the monitoring chamber 321a.

The pressure sensor frame 322 is stacked on one side of the monitoring chamber frame 321 and includes a pressure sensor 322a that measures the pressure in the monitoring chamber 321a through the communication space 321b of the monitoring chamber frame 321. The pressure sensor 322a is coupled to the lower surface of the pressure sensor frame 322 and is exposed to the monitoring chamber 221a through the communication space 321b of the monitoring chamber frame 321. The shape of the monitoring chamber frame 321 may be formed to correspond to the shape of the pressure sensor 322a, the pressure sensor 322a is exposed to the monitoring chamber 321a, the area or volume of the communication space 321b is minimized, and thereby wasted space may be minimized.

In addition, the pressure monitoring unit 320 may further include a pressure delay portion 323 that is in communication with the monitoring chamber 321a, allows the external pressure to act on the internal pressure of the monitoring chamber 321a, and delays an operation start of the external pressure for a predetermined time.

The pressure delay portion 323 includes a first opening 323a that is spaced apart from the pressure sensor 322a by a predetermined distance and formed in the pressure sensor frame 322, a second opening 323b that is formed in the monitoring chamber frame 321 and communicates between the first opening 323a and the monitoring chamber 321a, and a porous membrane 323c that is coupled to the first opening 323a.

Here, the second opening 323b is adjacent to the first opening 323a and is spaced apart from the communication space 321b by a predetermined distance, and the size of the second opening 323b is formed smaller than the size of the first opening 323a to prevent sudden pressure changes. As described above, the monitoring chamber 321a communicates with the outside using the first opening 323a and the second opening 323b, and the first opening 323a is sealed by the porous membrane 323c, so that the external pressure may also act on the internal pressure of the monitoring chamber 321a. In addition, the size of the second opening 323b is formed smaller than the size of the first opening 323a, the external pressure is reflected to the internal pressure of the monitoring chamber 321a, and a predetermined delay time occurs to prevent a rapid pressure change.

Next, additional configuration of the pressure monitoring unit 320 will be described.

The pressure sensor frame 322 may further include a circuit element (not illustrated), a memory 322b, and a communication module 322c.

A circuit element (not illustrated) is disposed around the pressure sensor 322a and the memory 322b stores the identification number or calibration information of the pump assembly 300, and may be coupled to the lower surface of the pressure sensor frame 322 together with the pressure sensor 322a. At this time, the shape of the communication space 321b may be formed to correspond to the shapes of the pressure sensor 322a and the memory 322b. The communication module 322c is coupled to one side of the pressure sensor frame 322 on the opposite side of the monitoring chamber frame 321, and transmits the pressure of the monitoring chamber 321a measured by the pressure sensor 322a and unique information about the pump assembly 300 contained in the memory 322b to the outside.

In addition, the monitoring chamber frame 321 may further include a trench 321c for coupling a sealing member that seals the coupling portion with the pressure sensor frame 322. The trench 321c is formed along the periphery of the monitoring chamber 321a on the opposite side, and the sealing member is disposed to seal the coupling portion with the pressure sensor frame 322. The coupling portion of the monitoring chamber frame 321 and the pressure sensor frame 322 may be sealed more firmly by the trench 321c.

Referring again to FIG. 3, the injection assembly 400 injects the drug discharged from the pump assembly 300 into the injection subject through the insertion tube. The insertion tube may be a needle or cannula.

The circuit unit 500 is equipped with electronic components necessary for driving and controlling the pump assembly 300, and includes a control unit that controls the operation of the drug injection apparatus 10.

FIG. 16 is a block diagram schematically illustrating the drug injection apparatus according to an embodiment of the present disclosure.

Based on the internal pressure of the pump assembly 300, a control unit 510 determines whether the path through which the drug in the pump assembly 300 moves or the flow path such as the insertion tube through which the drug of the injection assembly 400 moves is clogged. At this time, the pressure monitoring unit 320 is used to measure the internal pressure of the pump assembly 300.

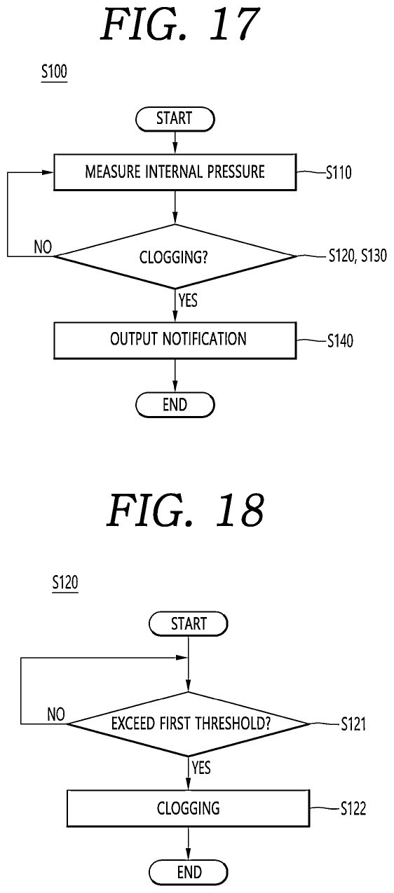

FIG. 17 is a flowchart for explaining a method for detecting clogging of the insertion tube according to an embodiment of the present disclosure. The method for detecting clogging of the flow path will be described with reference to FIG. 17.

A flow path clogging detection method S100 performed by the drug injection apparatus 10 includes measuring the internal pressure by the pump assembly 300 at every predetermined cycle (step S110), determining whether the flow path through which the drug moves is clogged based on the pressure measured by the control unit 510 (step S120 and step S130), and outputting a notification when it is determined that the flow path is clogged in step S 120 (step S140).

FIG. 18 is a flowchart for explaining an embodiment of the step of determining whether the flow path is clogged illustrated in FIG. 17. Referring to FIG. 18, the process of determining whether the flow path is clogged according to an embodiment (step S120) will be described.

The process of determining whether the flow path is clogged (step S120) includes determining whether the pressure measured in step S110 exceeds a first threshold (step S121). In step S121, when the measured pressure exceeds the first threshold, it is determined that the flow path is clogged (step S122).

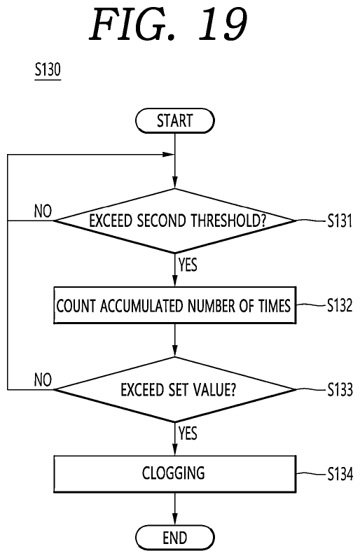

FIG. 19 is a flowchart for explaining another embodiment of the step of determining whether the flow path is clogged illustrated in FIG. 17. Referring to FIG. 19, the process of determining whether the flow path is clogged (step S130) according to another embodiment will be described.

The process of determining whether the flow path is clogged (step S130) includes determining whether a pressure difference between a pressure measured a first time point and a pressure measured a second time point exceeds a second threshold (step S131). When the pressure difference exceeds the second threshold in step S131, counting the accumulated number of times (step S132), and determining whether the accumulated number of times exceeds a set value (step S133). When the accumulated number of times exceeds the set value in step S133, it is determined that the flow path is clogged (step S134).

Additionally, the two embodiments of the step of determining whether the flow path is clogged described above may be used in combination. For example, when the pressure measured in step S121 does not exceed the first threshold, step S131 may be performed.

Those of ordinary skill in the technical field to which the present disclosure pertains will be able to understand that the present disclosure may be easily modified into other specific forms without changing its technical idea or essential features based on the above description. Therefore, the embodiments described above should be understood in all respects as illustrative and not restrictive. The scope of the present disclosure is indicated by the claims described below, and all changes or modified forms derived from the meaning and scope of the claims and their equivalent concepts should be construed as being included in the scope of the present disclosure.

The scope of the present application is indicated by the claims described below rather than the detailed description above, and all changes or modified forms derived from the meaning and scope of the claims and their equivalent concepts should be construed as being included in the scope of the present application.

1. A pump assembly of a drug injection apparatus, comprising:
an electroosmotic pump that includes a monitoring chamber that is a subject for pressure measurement, and electrochemically generates a positive pressure and a negative pressure alternately to suck and discharge a transfer subject fluid; and
a pressure monitoring unit that is coupled to one side of the monitoring chamber and measures an internal pressure of the monitoring chamber.

2. The pump assembly of claim 1, wherein the pressure monitoring unit includes:
a monitoring chamber frame that is coupled to one side of the monitoring chamber and forms a communication space;
a pressure sensor frame that is stacked on the monitoring chamber frame on the opposite side of the monitoring chamber; and
a pressure sensor that is coupled to the pressure sensor frame and measures a pressure of the monitoring chamber through the communication space of the monitoring chamber frame.

3. The pump assembly of claim 1, wherein
the electroosmotic pump includes a pressure generating portion that electrochemically generates the positive pressure and the negative pressure alternately; and a first isolation portion and a second isolation portion that are coupled to one side and the other side of the pressure generator, respectively, and
the monitoring chamber is formed between the first isolation portion and the pressure monitoring unit.

4. The pump assembly of claim 2, wherein
the pressure monitoring unit further includes a pressure delay portion that is in communication with the monitoring chamber, allows an external pressure to act on the internal pressure of the monitoring chamber, and delays an operation start of the pressure for a predetermined time.

5. The pump assembly of claim 4, wherein the pressure delay portion includes:
a first opening that is formed in the pressure sensor frame;
a second opening that is formed in the monitoring chamber frame and communicates between the first opening and the monitoring chamber; and
a porous membrane that is coupled to the first opening.

6. The pump assembly of claim 5, wherein
a size of the second opening is smaller than a size of the first opening.

7. The pump assembly of claim 5, wherein
the first opening of the pressure sensor frame is formed to be spaced apart from the pressure sensor by a predetermined distance,
the second opening of the monitoring chamber frame is formed adjacent to the first opening, and
the communication space of the monitoring chamber frame is formed to be spaced apart from the second opening, and formed in a region corresponding to the pressure sensor.

8. The pump assembly of claim 7, wherein
the communication space exposes the pressure sensor to the monitoring chamber and is formed such that an area or a volume of the communicating space is minimized.

9. The pump assembly of claim 2, wherein
the pressure sensor frame further includes a circuit element that is disposed around the pressure sensor.

10. The pump assembly of claim 2, wherein
the pressure sensor frame further includes a memory that stores the identification number or calibration information of the pump assembly.

11. The pump assembly of claim 2, wherein
the pressure sensor frame further includes a communication module that transmits the pressure measured by the pressure sensor to the outside.

12. The pump assembly of claim 11, wherein
the communication module is coupled to one side surface of the pressure sensor frame on the opposite side of the monitoring chamber frame.

13. The pump assembly of claim 2, wherein
the monitoring chamber frame is formed of a trench for coupling a sealing member that seals a coupling portion with the pressure sensor frame along a circumference thereof on the opposite side of the monitoring chamber frame.

14. A drug injection apparatus comprising:
the pump assembly according to claims 1; and
a control unit that detects clogging of a flow path of the drug injection apparatus based on an internal pressure of the pump assembly measured in the pump assembly.

15. A method of detecting clogging of a flow path of a drug injection apparatus, the method comprising:
measuring an internal pressure of a pump assembly at predetermined intervals; and
determining whether the flow path of the pump assembly is clogged based on the internal pressure measured in the pump assembly by a control unit.

16. The method of claim 15, wherein the determining whether the flow path is clogged includes:
determining whether the measured pressure exceeds a first threshold; and
determining that clogging of the flow path occurs when the measured pressure exceeds the first threshold.

17. The method of claim 15, wherein the determining whether the flow path is clogged further includes:
determining whether a difference between a pressure measured at a first time point and a pressure measured at a second time point exceeds a second threshold;
counting the accumulated number of times when the pressure difference exceeds the second threshold;
determining whether the accumulated number of times exceed a set value; and
determining that clogging occurs when the accumulated number of times exceeds the set value.
